(19) United States
(12) Patent Application Publication
(10) Pub. No.: US 2005/0228216 A1
(43) Pub. Date: Oct. 13, 2005

(54) Method for treating gastroesophageal reflux disease

(76) Inventor: Jan Albert de Vries, Zelhem (NL)

Correspondence Address: Melvin I. Stoltz, 51 Cherry Street, Milford, CT, 06460, US

(21) Appl. No.: 11/084,918

(22) Filed: Mar. 21, 2005

Related U.S. Application Data
(60) Provisional application No. 60/560,393, filed on Apr. 8, 2004.

(51) Int. Cl. A61F 2/00; A61B 5/05

(52) U.S. Cl. 600/29

(57) ABSTRACT

By providing an elastic form stable material which is capable of being delivered directly into the body tissue in the vicinity of or surrounding the lower esophagus of a living creature, gastroesophageal reflux disease is able to be effectively treated. In the preferred method, the elastic form stable material is injected directly into lower esophagus and allowed to cure in situ, thereby achieving the desired result.

### RELATED APPLICATIONS

This application is related to U.S. Provisional Patent Application Ser. No. 60/560,393, filed Apr. 8, 2004 entitled REFLUX.

### TECHNICAL FIELD

The present invention relates to a method of treating gastroesophageal reflux disease (abbreviated to GERD) in a mammal, in particular a human being. This invention also relates to a composition suitable for use in such a method, its preparation and use.

### BACKGROUND ART

Gastroesophageal reflux disease or GERD relates to the repeated backup of food and digestive fluid from the stomach into the esophagus. Symptoms thereof comprise heartburn and acid indigestion. GERD is caused by malfunctioning of the lower esophageal sphincter, a small ring of muscles. GERD, if not treated, can lead to serious disorders or complications, such as bleeding and narrowing of the esophagus.

GERD can be treated in a variety of ways ranging from changing lifestyle, treatment by drugs, prothesis and surgery. An alternative to surgery, e.g. known from WO 03/072196 involves modifying the lower esophagus by injecting an emulsion of microparticulate material in a suitable liquid carrier into body tissue sites surrounding the esophagus, in particular the submuscosal tissue site adjacent to or in the vicinity of the lower esophageal sphincter (hereinafter abbreviated to LES).

In employing these prior art methods, it has been found that the dimensions of the microparticulate material are critical. The particles should be able to pass through a hypodermic needle, while after injection they should substantially remain at the injection site. However, careful selection of suitable dimensions for the microparticulate material is required and, even when care is exercised, the risk remains that these particles are susceptible to migration.

In addition, it has been found that use of prior art methods typically suffer from uncertainty and unpredictability of effective results. Furthermore, a system which provides results which are dependable, repeatable, and reproducible has long been sought in this industry and has eluded prior art developments.

Therefore it is the principal object of the present invention to provide a method for treating gastroesophageal reflux disease in a mammal, in particular a human being, wherein a highly effective, dependable, and repeatable results are realized.

Another object of the present invention is to provide a method for treating gastroesophageal reflux disease, having the characteristic features described above, which is easily tolerated by patients, while also providing predictable results.

Another object to the present invention is to provide a method for treating gastroesophageal reflux disease, having the characteristic features described above, which virtually eliminates the risk of having microparticulate materials migrate away from an injection site.

Other and more specific objects will in part be obvious and will in part appear hereinafter.

### SUMMARY OF THE INVENTION

By employing the present invention, all of the difficulties and drawbacks of the prior art are eliminated, and a method for treating gastroesophageal reflux disease in a living creature, in particular a human being, is realized. In accordance with the present invention, this desired result is attained by injecting a curable elastomer precursor composition into the body tissue in the vicinity of, or surrounding, the lower esophagus, and allowing the composition to cure to an elastic form stable material.

In the preferred method of the present invention, an elastomer precursor is prepared, and subsequently injected in one or more injection sites adjacent to or in the vicinity of the LES. After injection the composition cures to an elastic form stable material.

The cured material, which is a solid mass, holds its initial position, and the risk of migration is significantly reduced. The absence of microparticulates in the material used in the invention also contributes to the reduction of the risk of migration.

The form stability of the material of the preferred invention ensures that the respective tissue is sufficiently strengthened in order to enhance the appropriate operation of the LES, thereby preventing reflux of the content of the stomach, back into the esophagus. Nevertheless, a patient treated according to the invention is still able to vomit naturally, because of the elasticity of the form stable material. Non-elastic material would complicate such vomiting severely. Simultaneously the form stable material itself is flexible enough to adapt to body movements, so that a patient treated according to the invention does not experience any discomfort.

The invention also relates to a method of preparing a composition for the treatment of gastroesophageal reflux disease in a mammal, in particular a human being, comprising a curable elastomer-precursor composition.

A further aspect of the invention concerns the use of elastic form stable material, preferably a curable elastomer-precursor composition, in the treatment of gastroesophageal reflux disease in mammals, in particular human beings.

The invention also relates to the use of an elastic form stable material, preferably a curable elastomer-precursor composition in the preparation of a treating agent for the treatment of gastroesophageal reflux disease in a mammal, in particular a human being.

The invention of accordingly comprises the several steps and the relation of one of more such steps with respect to each of the others, and the composition possessing the features, properties, and relation of elements, which will be exemplified in the composition hereinafter described, and the scope of the invention will be indicated in the claims.

### DESCRIPTION OF THE PREFERRED EMBODIMENTS

In accordance with the preferred embodiment of the present invention, the elastic form stable material comprises an elastomer-precursor composition which is cured in situ. That is to say, the elastomer-precursor is prepared in advance and then applied to the appropriate positions by suitable equipment. Preferably the composition is injected in at least three peripheral positions surrounding the LES. More preferably several injections are carried out in such a way that the cured composition completely surrounds the esophagus, in particular at the LES, as an annular support.

Commercially available medical grade silicone elastomers are preferred materials for use as polymer precursor in this composition. A more preferred material is a poly (dimethyl siloxane) such as hydroxyl-end-blocked poly (dimethyl siloxane). Such silicone elastomers of medical grade as pourable, multi-component silicone are commercially available. Typically, these silicone elastomers are fast curing materials.

For these types of elastomers, propyl orthosilicate is a useful cross-linking agent. Fillers and diluents (medicinal fluids such as known under the trade name Dimeticonum) in order to reduce viscosity may be added as needed.

In the preferred formulation, an initiator like tin (II) octoate or platinum initiates the polymerization reaction with splitting of propanol. The reaction proceeds without the generation of sensible heat. Silphenylene polymer can be used in a similar way.

In order to be able to trace the position of the applied composition by X-ray monitoring, a preferred embodiment of the composition comprises a radiopaque material such as silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents. In this regard, silver powder is the preferred radiopaque material, since smaller proportions are needed in the composition and the material also provides a material anti-bacterial and anti-microbial activity.

A composition as explained above is known, per se, as a material suitable for the non-surgical, reversible sterilization of females. In this known sterilization method, the composition is injected in the oviduct portion adjacent the uterus, where it forms in situ a block or plug in the oviduct, thereby preventing the passage of ovum from the ovaries to the uterus and sperm from entering the oviduct and thus conception. See e.g. U.S. Pat. No. 4,245,623. However, the present invention is patentably distinguishable from the prior art.

A preferred composition for use in the method according to the invention comprises about 60-85% by weight poly (dimethyl siloxane), about 2-5% cross-linking agent, a diluent in the range of 10-20% and about 10-20% radiopaque powder. An even more preferred composition comprises about 68 wt. % poly (dimethyl siloxane), about 4% cross-linking agent, about 13% dimeticonum and about 16% silver powder.

In the method according to the invention the composition is advantageously prepared in advance in a mixing-dispensing device. Such a device, wherein the function of mixing the components is combined with the function of dispensing the thus prepared mixture is known per se, e.g. from the above U.S. Patent, the content of which is incorporated in its entirety by reference.

As already indicated above, the invention also relates to a method of preparing a composition for the treatment of gastroesophageal reflux disease in a mammal, in particular a human being, comprising a curable elastomer-precursor composition. In addition, other mixing and dispensing systems are known in the art.

One of these alternate, two-component mixing and dispensing devices is fully disclosed in pending U.S. patent application Ser. No. 11/000,578, filed Nov. 30, 2004, entitled Two Component Mixing and Dispensing Device. If desired, the composition and method of the present invention may be employed using this device and the pertinent disclosure of this pending patent application is incorporated herein by reference.

As already indicated above, this invention also relates to a method of preparing an injection composition for injection into the lower esophagus of a living creature, in particular a human being, said composition comprising an elastic form stable material. The above-mentioned preferred features of the treatment method according to the invention are similarly applicable to the preparation method according to the invention.

Advantageously the composition is packaged as a kit of parts, comprising at least a first container filled with an elastomer precursor and optionally a diluent, and a second container filled with a cross-linking agent for this elastomer precursor. More preferably, the composition is packaged in a mixing-dispensing device, comprising such containers and a temporary seal between the containers, wherein one of these containers is provided with a stirrer which can be operated manually or powered by an external source.

An example of such a device is also known from the above-mentioned U.S. Patent. Devices of this type can be used for injecting the thus prepared precursor composition by connecting a suitable flexible tube to the container acting as a mixing chamber and providing an appropriate needle at the other end of the tube.

In the invention a flowable composition is prepared from the various components, preferably in a combined mixing-dispensing device as explained above, and then immediately used. The patient is prepared for the treatment according to standard medical procedures. A catheter is brought via the mouth into the esophagus until the injection needle reaches the vicinity of the LES. When this position is reached, the wall of the esophagus is punctured by the injection needle. Then, the composition is forced from the respective container via a suitable flexible tube to the needle and deposited directly adjacent this wall in the respective tissue.

In order to more accurately position the deposit of the composition, preferably the needle is not open at the tip but has more exits at the side face directly adjacent to the tip. Use of this modified needle allows the surgeon to position the composition more easily adjacent the esophagus wall into the tissue. Thereafter the needle is retracted and the composition is allowed to cure in situ. As already mentioned, preferably the composition is applied at multiple locations surrounding the esophagus. In view thereof the above actions can be repeated as needed.

It will thus be seen that the objects set forth above, among those made apparent from the preceding description, are efficiently attained and, since certain changes may be made in carrying out the above method and in the composition set forth without departing from the scope of the invention, it is intended that all matter contained in the above description shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

Particularly, it is to be understood that in said claims, ingredients or compounds recited in the singular are intended to include compatible mixtures of such ingredients wherever the sense permits.

1. A method for treating gastroesophageal reflux disease in a mammal, in particular a human being, comprising the step of injecting a curable elastomer-precursor composition into the body tissue in the vicinity of or surrounding the lower esophagus, and allowing the composition to cure to an elastic form stable material.

2. The method defined in claim 2, wherein said composition is injected at least in three positions.

3. The method defined in claim 1, wherein said composition comprises a silicone elastomer.

4. The method defined in claim 3, wherein said silicone elastomer is poly (dimethyl siloxane).

5. The method defined in claim 3, wherein said composition further comprises a cross-linking agent and a diluent.

6. The method defined in claim 1, wherein said composition comprises a radiopaque material.

7. The method defined in claim 6, wherein said radiopaque material comprises one selected from the group consisting of silver powder, barium sulfate, bismuth trioxide, zirconium dioxide, tantalum or titanium powders or fibers, calcium sulfate, calcium phosphate, hydroxyapetite, tri calcium phosphate, and other medically appropriate opacifier agents.

8. The method defined in claim 7, wherein curable bone filler material is further defined as comprising:
A. between about 60% and 85% by weight based upon the weight of the entire composition of poly (dimethyl siloxane);
B. between about 2% and 5% by weight based upon the weight of the entire composition of the cross-linking agent;
C. between about 100% and 20% by weight based upon the weight of the entire composition of the diluent; and
D. between about 100% and 20% by weight based upon the weight of the entire composition of the radiopaque material.

9. the method defined in claim 1, wherein said composition is prepared in advance in a mixing-dispensing device.

10. The method of preparing a composition for the treatment of gastroesophageal reflux disease in a mammal, in particular a human being, said composition comprising an elastic form stable material consisting of a curable elastomer-precursor composition and additive intermixed therewith and prepared in advance in mixing-dispensing device.

11. The method defined in claim 10, wherein the curable elastomer-precursor composition comprises a silicone elastomer.

12. The method defined in claim 11, wherein said silicone elastomer-is poly (dimethoxy siloxane).

13. The method defined in claim 11, wherein said additives are further defined as comprising a cross-linking agent and a diluent.

14. The method defined in claim 10, wherein said composition comprises a radiopaque material.

15. The method defined claim 13, wherein said composition is packaged as a kit of parts, comprising filling a first container with said silicone elastomer, and filling a second container with said cross-linking agent.

16. The method defined in claim 15, wherein said kit of parts comprises a mixing-dispensing device, and said container comprises a temporary seal between the containers, wherein one container is provided with a movable stirrer.

17. The method defined in claim 14, comprising the steps of thoroughly intermixing:
A. between about 60% and 85% by weight based upon the weight of the entire composition of poly (dimethyl siloxane);
B. between about 2% and 5% by weight based upon the weight of the entire composition of the cross-linking agent;
C. between about 100% and 20% by weight based upon the weight of the entire composition of the diluent; and
D. between about 100% and 20% by weight based upon the weight of the entire composition of the radiopaque material, thereby forming the desired composition.

18. Use of an elastic form stable material, preferably a curable elastomer-precursor composition in the preparation of a treating agent for the treatment of gastroesphageal reflux disease in a mammal, in particular a human being.
